(19) United States
(12) Patent Application Publication
(10) Pub. No.: US 2020/0069862 A1
(43) Pub. Date: Mar. 5, 2020

(54) SPLASHBACK SHIELD FOR WOUND CLEANSING

(71) Applicant: Mike Petrich, Highland, CA (US)

(72) Inventor: Mike Petrich, Highland, CA (US)

(21) Appl. No.: 16/120,399

(22) Filed: Sep. 3, 2018

(51) Int. Cl. A61M 3/02

(52) U.S. Cl. CPC A61M 3/0287; A61M 3/0262

(57) ABSTRACT

A splashback shield for connection to a saline bottle containing a saline solution. The splashback shield has a neck with internal threads. A shower barrier is positioned adjacent to the neck. The shower barrier has a plurality of holes. A shield section is positioned adjacent to the shower barrier. A plurality of drains is cut into the shield section. The plurality of drains permits the drainage of the saline solution after the patient's wound has been irrigated.

The present invention relates to medical safety devices, and in particular, to devices for preventing exposure to contaminated saline solution during wound cleansing procedures.

### BACKGROUND OF THE INVENTION

For a patient that has suffered a wound, it is very important that the wound is properly cleansed. Foreign matter, bacteria and other contaminates must be removed to ensure proper healing and minimize risk of infection. It is also important to clean and irrigate the wounded area in order to achieve proper visualization of the wound and surrounding tissues.

To clean a wound a saline solution is typically squirted onto the wound area. For example, FIGS. 1-3 show images of prior art saline solution bottle 1 containing a saline solution. A saline solution is a mixture of salt and water. Normal saline solution contains 0.9 percent sodium chloride (salt), which is similar to the sodium concentration in blood and tears. Bottle 1 includes cap 2 which is threaded onto threads 3 of bottle 1. A spray nozzle (not shown) is typically threaded onto threads 3 to facilitate spraying of a patient's wound for cleaning purposes.

When spraying a wound, there is a danger of splashback to the personnel providing medical treatment. For example, medical personnel are worried that the saline solution will splash back onto them after it has been squirted onto the wound. As it splashes back, the saline solution may also contain blood and tissue from the patient. If the patient's blood is infected with a dangerous virus (for example, the HIV virus or the Hepatitis B virus) the health care worker could be in real danger of infection.

The prior art has provided examples of splashback shields that are designed to protect the health care worker from splashback during wound irrigation. However, the prior art splashback shields tend to be costly, overly complicated and impractical.

What is needed is a better splashback shield to prevent splashback of potentially infectious fluids and body tissues during wound irrigation.

### SUMMARY OF THE INVENTION

The present invention provides a splashback shield for connection to a saline bottle containing a saline solution. The splashback shield has a neck with internal threads. A shower barrier is positioned adjacent to the neck. The shower barrier has a plurality of holes. A shield section is positioned adjacent to the shower barrier. A plurality of drains is cut into the shield section. The plurality of drains permits the drainage of the saline solution after the patient's wound has been irrigated.

### BRIEF DESCRIPTION OF THE DRAWINGS

FIGS. 1-3 show a prior art saline bottle.

FIGS. 4-7 show a preferred embodiment of the present invention.

FIGS. 8-10 show a preferred method for utilizing a preferred embodiment of the present invention.

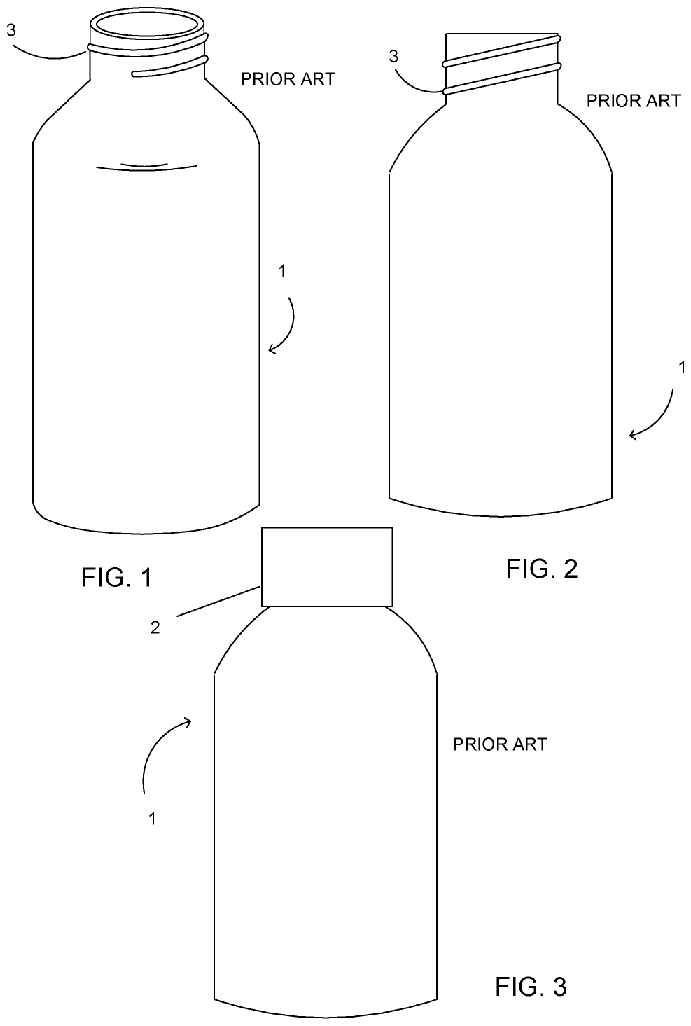

### DETAILED DESCRIPTION OF THE PREFERRED EMBODIMENTS

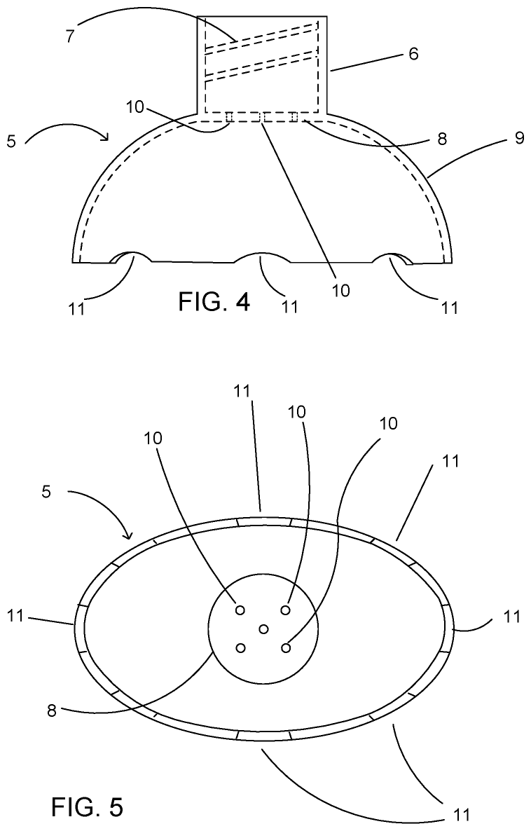

FIG. 4 shows a side view of splashback shield 5 and FIG. 5 shows a bottom view of splashback shield 5. In a preferred embodiment splashback shield 5 is fabricated from plastic. Neck 6 includes internal threads 7. Internal threads 7 mesh with threads 3 of saline solution bottle 1. Shower barrier 8 is positioned between neck 6 and shield section 9. Shower barrier 8 includes multiple holes 10. In a preferred embodiment, shower barrier 8 includes five holes, each having a diameter or approximately 20 gage (20 gage=0.81 mm).

Shield section 9 is roughly semi-spherical with an elliptical cross section, as shown in FIGS. 4 and 5. The bottom edge of shield section 9 includes multiple drains 11.

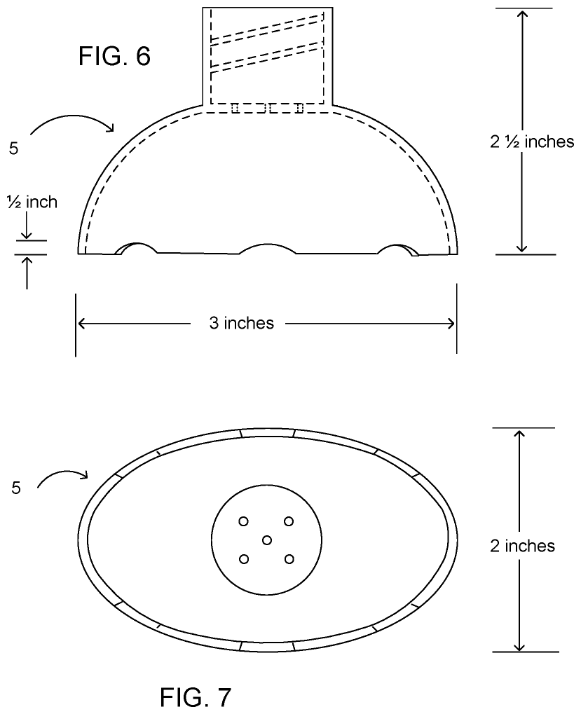

It should be noted that the exact dimensions of splashback shield 5 can be varied depending upon the wishes and needs of the user. The dimensions of one preferred splashback shield 5 is shown in FIGS. 5 and 6.

### Preferred Method for Utilizing Splashback Shield

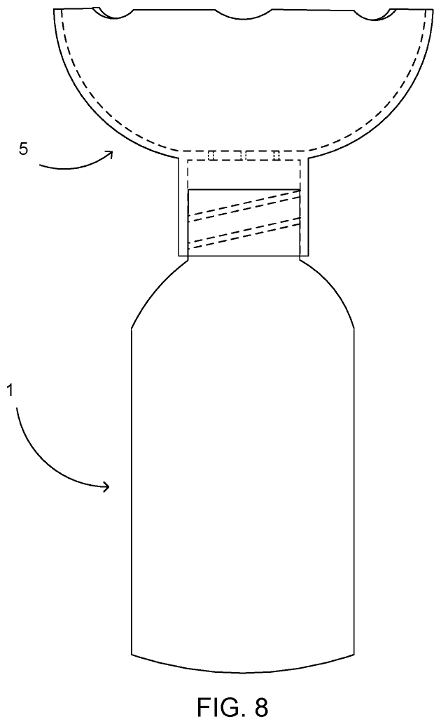

FIGS. 8-10 illustrate a preferred method for utilizing splashback shield 5.

In FIG. 8, the user has threaded splashback shield 5 onto the top of bottle 1 containing a saline solution.

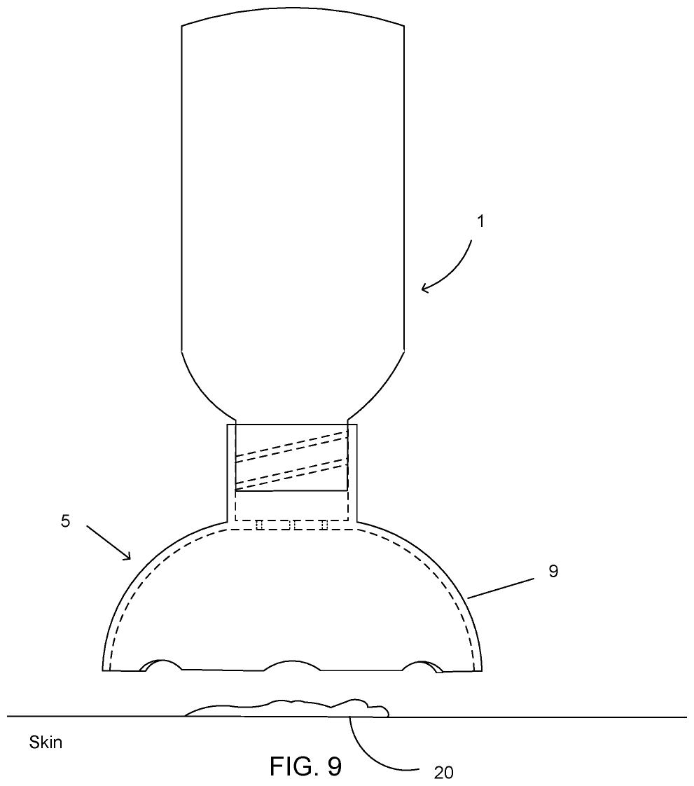

In FIG. 9 the user has flipped saline bottle 1 over so that shield section 9 of splashback shield 5 is positioned over wound 20 of the patient.

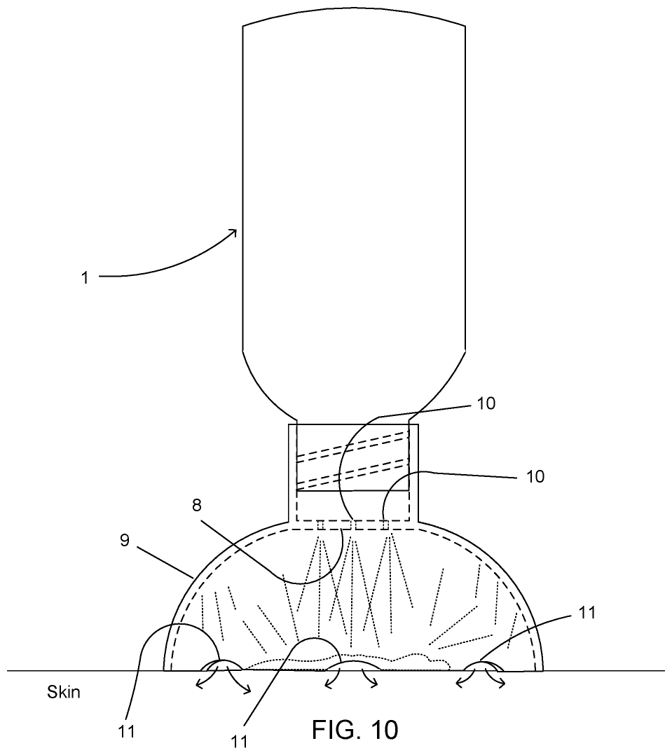

In FIG. 10 the user has lowered splashback shield 5 so that shield section 9 is resting on the surface of the patient's skin. Shield section 9 is surrounding wound 20, as shown. The user squeezes bottle 1, causing the saline solution contained within bottle 1 to spray outward through holes 10 of shower barrier 8. The saline solution sprays outward through holes 10 and showers down upon wound 20. Saline solution that may also include blood or tissue from wound 10 is then directed upward from the wound, but is contained within shield section 9. Excess saline solution flows outward from the contained area through drains 11.

Although the above-preferred embodiments have been described with specificity, persons skilled in this art will recognize that many changes to the specific embodiments disclosed above could be made without departing from the spirit of the invention. Therefore, the attached claims and their legal equivalents should determine the scope of the invention.

What is claimed is:

1) A splashback shield for connection to a saline bottle containing saline solution, comprising:
A) a neck having internal threads,
B) a shower barrier positioned adjacent to said neck, said shower barrier having a plurality of holes,
C) a shield section positioned adjacent to said shower barrier, and
D) a plurality of drains cut into said shield section, said plurality of drains for permitting the drainage of said saline solution after it has irrigated a patient's wound.

2) The splashback shield as in claim 1, wherein said splashback shield is fabricated from plastic.

3) The splashback shield as in claim 1, wherein said internal threads of said neck mesh with threads on said saline bottle.

4) The splashback shield as in claim 1, wherein said plurality of holes in said shower barrier is five twenty-gage holes.

5) The splashback shield as in claim 1, wherein said shield section is semi-spherical with an elliptical cross section.

6) A method for irrigating a patient's wound, comprising the steps of:
A) obtaining a splashback shield for connection to a saline bottle containing saline solution, said splashback shield comprising:
i) a neck having internal threads,
ii) a shower barrier positioned adjacent to said neck, said shower barrier having a plurality of holes,
iii) a shield section positioned adjacent to said shower barrier, and
iv) a plurality of drains cut into said shield section, said plurality of drains for permitting the drainage of said saline solution after it has irrigated a patient's wound,
B) connecting said splashback shield to said saline bottle containing saline solution,
C) squeezing said saline bottle so that said saline solution is showered down onto said patient's wound,
D) containing said saline solution within said shield section after said saline solution contacts and irrigates said wound, and
E) draining said saline solution through said plurality of drains.

7) The method as in claim 6, wherein said splashback shield is fabricated from plastic.

8) The method as in claim 6, wherein said internal threads of said neck mesh with threads on said saline bottle.

9) The method as in claim 6, wherein said plurality of holes in said shower barrier is five twenty-gage holes.

10) The method as in claim 6, wherein said shield section is semi-spherical with an elliptical cross section.
